(19) United States
(12) Patent Application Publication
(10) Pub. No.: US 2019/0230970 A1
(43) Pub. Date: Aug. 1, 2019

(54) COMPOSITIONS AND METHODS FOR THE PREVENTION AND TREATMENT OF COLITIS IN INFANTS

(71) Applicant: Adam Hanna, Edmond, OK (US)

(72) Inventor: Adam Hanna, Edmond, OK (US)

(21) Appl. No.: 16/375,776

(22) Filed: Apr. 4, 2019

Related U.S. Application Data
(63) Continuation of application No. PCT/US17/55528, filed on Oct. 6, 2017.
(60) Provisional application No. 62/404,927, filed on Oct. 6, 2016.

(51) Int. Cl. A23L 33/00; A61K 38/44; A61K 35/20

(52) U.S. Cl. CPC A23L 33/40; A61K 38/44; A23V 2200/32; C12Y 104/03022; A23V 2002/00; A61K 35/20

(57) ABSTRACT

This disclosure describes compositions and methods for preventing and/or treating necrotizing enterocolitis (NEC) and preventing loss of intestinal integrity and/or improving intestinal integrity in neonatal subjects. Compositions useful in the methods of the disclosure comprise diamine oxidase (DAO) which can be from any source and formulated for systemic administration.

### CROSS-REFERENCE TO RELATED APPLICATIONS

This application is a continuation of International Application No. PCT/US2017/055528, filed Oct. 6, 2017, which claims priority to U.S. Provisional Application No. 62/404,927, filed Oct. 6, 2016, the entirety of which are incorporated herein by reference in their entirety.

### BACKGROUND

Necrotizing enterocolitis (NEC) is a major cause of morbidity and mortality in premature infants including low birth weight infants (<2500 grams). NEC is characterized by intestinal inflammation that often progresses to systemic infection, organ failure and death. Approximately 15-20% of infants that weigh less than 2500 grams at birth will develop NEC during their first few weeks of life. Approximately 20% of low-birth-weight babies who develop NEC will die, despite receiving currently used supportive treatments. Term infants can also develop NEC. Currently, there are no FDA approved medications for treating or preventing NEC.

Although mild cases of NEC can be treated medically, more severe cases require surgical resection of necrotic bowel—leaving many surviving infants unable to absorb sufficient nutrition without intensive support therapy. A case of medically-managed NEC will increase a premature infant's cost of treatment by an average of $74,004, with the additional cost of treatment increasing to $198,040 for surgically-managed NEC. Even with appropriate treatment, NEC mortality rates remain extremely high at approximately 20-30%. Therefore, there is a need for compositions and methods to prevent and treat NEC to reduce mortality, life-long medical complications, and the cost of treatment.

The present disclosure provides compositions and methods for the prevention and treatment of colitis and loss of intestinal integrity in premature infants and term infants that address these unmet needs. In particular, compositions and methods useful in the treatment or prevention of NEC are provided.

### SUMMARY

The present disclosure is directed to compositions and methods for the prevention and treatment of colitis in infants. In particular, the compositions and methods of the present disclosure are useful in the treatment and prevention of NEC. The compositions and methods provided can be used to prevent or treat colitis, including NEC, and to prevent the loss of or improve intestinal integrity in such infants.

In one embodiment, a method for preventing or treating colitis is provided. The method comprises administering a therapeutically effective amount of diamine oxidase to a subject suffering from colitis or at risk for developing colitis. The subject may be a premature infant at risk for developing NEC. The administration step may be practiced by feeding the infant breast milk or infant formula that is supplemented with a therapeutically effective amount of diamine oxidase. A therapeutically effective amount, with respect to diamine oxidase, in all instances is an amount that is greater than the amount of diamine oxidase that may be naturally present in breast milk or greater than the amount found in standard infant formulas.

In an embodiment, a method for preventing colitis includes the steps of adding diamine oxidase (DAO) (ExPASy Bioinformatica Resource Portal entry EC 1.4.3.22) to human expressed breast milk to yield a DAO-supplemented breast milk and administering the DAO-supplemented breast milk to an infant. The DAO can be added to the human expressed breast milk as part of a human milk fortifier.

In another embodiment, a method for treating colitis includes the steps of adding diamine oxidase (DAO) to human expressed breast milk to yield a DAO-supplemented breast milk and administering the DAO-supplemented breast milk to an infant. The DAO can be added to the human expressed breast milk as part of a human milk fortifier.

In still another embodiment, a method for preventing loss of intestinal integrity in an infant includes the steps of adding diamine oxidase (DAO) to human expressed breast milk to yield a DAO-supplemented breast milk and administering the DAO-supplemented breast milk to an infant. The DAO can be added to the human expressed breast milk as part of a human milk fortifier.

In yet another embodiment, a method for improving intestinal integrity in an infant includes the steps of adding diamine oxidase (DAO) to human expressed breast milk to yield a DAO-supplemented breast milk and administering the DAO-supplemented breast milk to an infant. The DAO can be added to the human expressed breast milk as part of a human milk fortifier.

In an embodiment, a method for preventing colitis includes the steps of administering a composition comprising DAO to an infant, where the composition does not include human expressed breast milk.

In another embodiment, a method for treating colitis includes the steps of administering a composition comprising DAO to an infant, where the composition does not include human expressed breast milk.

In still another embodiment, a method for preventing loss of intestinal integrity in an infant includes the steps of administering a composition comprising DAO to said infant, where the composition does not include human expressed breast milk.

In yet another embodiment, a method for improving intestinal integrity in an infant includes the steps of administering a composition comprising DAO to said infant, where the composition does not include human expressed breast milk.

In an embodiment, a composition is provided comprising human expressed breast milk and DAO, where the DAO is present at a non-physiological concentration.

In another embodiment, a composition is provided comprising a human milk fortifier and DAO.

In another embodiment, a composition is provided comprising an infant formula and DAO.

In another embodiment, a method comprises manufacturing infant formula or human milk fortifier, wherein the infant formula or human milk fortifier comprises DAO. The method can further include a step of adding DAO to the infant formula or human milk fortifier.

### BRIEF DESCRIPTION OF THE DRAWINGS AND FIGURES

The patent or application file contains at least one drawing executed in color. Copies of this patent or patent application publication with color drawing(s) will be provided by the Office upon request and payment of the necessary fee.

In the Figures, the use of asterisks is understood to indicate the level of a statistically significant difference between values. Where bars labeled with asterisks are used, the statistically significant difference is between the values at the ends of each bar. The term “ns” indicates a non-statistically significant difference.

FIG. 1 shows the effect of DAO on survival in the Dithizone/Klebsiella Necrotizing Enterocolitis (DK NEC) model.

FIG. 2A shows the representative hematoxylin and eosin sections of the intestine from sham (n=6), NEC group (n=10), and NEC+DAO (n=14).

FIG. 2B shows assessment of intestinal injury scores in the different groups depicted in FIG. 2A.

FIG. 3 shows the effect of DAO on intestinal permeability in the DK NEC model.

FIGS. 4A-4F shows the effect of DAO on intestinal levels of pro-inflammatory and anti-inflammatory cytokines in the DK NEC model.

FIG. 4A shows the effect of DAO on the intestinal level of GRO-α.

FIG. 4B shows the effect of DAO on the intestinal level of IL-1β.

FIG. 4C shows the effect of DAO on the intestinal level of IL-12p70.

FIG. 4D shows the effect of DAO on the intestinal level of IL-6.

FIG. 4E shows the effect of DAO on the intestinal level of IFNγ.

FIG. 4F shows the effect of DAO on the intestinal level of IL-10.

FIG. 5 depicts a timeline of onset of NEC in mouse models.

FIG. 6 shows survival in the DK NEC model.

FIG. 7 shows the intestinal permeability measured by serum FITC-dextran.

FIG. 8A shows tissue cytokine expression of IL-1β.

FIG. 8B shows tissue cytokine expression of IL-6.

FIG. 8C shows tissue cytokine expression of IL-10.

FIG. 9 shows the representative histological samples in the sham mouse model, NEC mouse model, and NEC mouse model treated with DAO.

FIG. 10A shows the blinded histology NEC grade assigned to the sham mouse model, NEC mouse model, and NEC mouse model treated with DAO.

FIG. 10B shows serum histamine concentrations for the sham mouse model, NEC mouse model, and NEC moue model treated with DAO.

### DETAILED DESCRIPTION OF THE INVENTION

The present disclosure provides compositions and methods for the prevention of and treatment of colitis and preventing the loss of and/or improving intestinal integrity in infants. In particular, compositions and methods of the present disclosure can be useful in the treatment or prevention of NEC in infants. More particularly, the compositions and methods of the present disclosure can be useful in the treatment or prevention of NEC in premature infants.

Circulatory DAO levels in premature infants decrease to baseline levels about 12 days after birth. There is no observed, statistically significant difference in circulating DAO levels in premature infants versus term infants and no correlation has been found between circulating DAO levels and NEC. It has surprisingly been found that, despite the foregoing, administration of DAO to infants can prevent or treat colitis. In particular, administration of DAO to infants can prevent or treat NEC and prevent loss of intestinal integrity or improve intestinal integrity in such infants.

### Definitions

Certain terminology is used in the following description for convenience only and is not limiting. Certain words used herein designate directions in the drawings to which reference is made. Unless specifically set forth herein, the terms “a,” “an” and “the” are not limited to one element, but instead should be read as meaning “at least one.” As used herein “another” means at least a second or more. The terminology includes the words noted above, derivatives thereof and words of similar import.

The use of the term “or” in the claims is used to mean “and/or” unless explicitly indicated to refer to alternatives only or the alternatives are mutually exclusive.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

All terms used herein, unless otherwise specified, will have their commonly understood plain, scientific meaning.

“DAO” refers to diamine oxidase obtained from any source. By way of example, but not limitation, DAO can be obtained from human, plant, animal and microorganism sources and can be recombinantly produced in any appropriate organism.

“Plant origin” refers to all DAO obtained from plant organisms.

“Non-plant origin” refers to all DAO not derived from plants but from animal organisms or other non-plant organisms. Thus, this definition covers all isolated DAO from living organisms other than plants. By way of example, but not limitation, non-plant origin would include DAO derived from microorganisms as well as from mammals such as pigs.

“Animal origin” refers to all DAO derived from animal organisms.

“Biotechnological origin” means all DAO recombinantly prepared in cell culture or any other protein expression systems. Any protein expression system can be used including, by way of example but not limitation, cell culture, insect-based systems, plant-based systems and animal-based systems.

“Non-human origin” refers to all DAO that is obtained from a source other than a human being.

As used herein, “prevention” or “preventing” refers to preventing the onset of a disease or pathological condition. For example, this disclosure provides methods and compositions for preventing the onset of NEC in infants and for preventing or attenuating the loss of intestinal integrity in such infants.

“Treatment” and “treating”, as used herein, refer to clinical intervention to alter a disease or other condition and can include a cure or partial cure of the disease or condition, elimination and/or reduction of symptoms, elimination or reduction of pathological consequences of the disease or condition, and reduction of the severity of the disease or condition.

“Physiological concentration” refers to a concentration of DAO within the expected ranges found naturally in human expressed breast milk. DAO concentration (in terms of activity) on the first day of lactation is 0.186+/−0.02 Units/mL, on the second day of lactation is 0.175+/−0.02 Units/mL, and by the 30th day of lactation is 0.139+/−0.026 Units/mL, thus the range of physiological concentration of DAO, as used in the present disclosure is between 0.1364-0.188 Units/mL. The “maximum physiological concentration” in breast milk is, for purposes of the present disclosure, 0.188 Units/mL.

“Non-physiological concentration” refers to a concentration of DAO that exceeds the physiological concentration through addition of exogenous DAO. For example, by adding exogenous DAO to a sample of human expressed breast milk, the total concentration of DAO will be greater than the physiological concentration of DAO in the human expressed breast milk (prior to addition of exogenous DAO) and, thus, at a non-physiological concentration.

“Fetal physiological concentration” refers to the concentration of DAO to which a premature infant would be exposed to from the placenta and amniotic fluid if the premature infant was still gestating.

“Human milk fortifier” refers to any composition which is added to human expressed breast milk to supplement or otherwise fortify the human expressed breast milk. By way of example but not limitation, human milk fortifiers can include commercially available preparations such as SIMILAC human milk fortifier, PROLACTA human milk fortifier, and ENFAMIL human milk fortifier.

Infants and Premature Infants

A “premature infant” is understood to refer to a human child that is born prematurely as compared to the normal gestational term. In some embodiments, a premature infant is a low birth weight infant weighing less than 2500 grams. In some embodiments, a premature infant is a low birth weight infant weighing less than 1500 grams.

The term “infant”, absent modification by the terms “premature” or “term” is intended to include all infants, as the term is commonly understood, including both “premature” and “term” infants.

In some embodiments, a subject is a premature infant. In certain embodiments, the premature infant is suffering from colitis. In some embodiments, the premature infant is suffering from NEC.

In some embodiments, a subject is a term infant. In certain embodiments, the term infant is suffering from colitis. In some embodiments, the term infant is suffering from NEC.

In such embodiments, the premature infant or term infant can be treated using the methods and compositions of the present disclosure.

In some embodiments, the infant is a neonate, which is an infant within 28 days of birth regardless of the timing of birth with respect to the normal gestational term. In some embodiments, the infant is within one year of birth.

Colitis

Compositions and methods of the present disclosure can be useful in the prevention or treatment of colitis in infants. Colitis includes, by way of example, but not limitation, includes NEC, food protein-induced proctocolitis (also known as allergic or eosinophilic proctocolitis or “protein intolerance”), benign dietary protein proctitis, eosinophilic proctitis/colitis, breast milk-induced colitis, cows milk colitis, infectious colitis (including due to to C. diff., Salmonella, rotavirus and the like), ischemic colitis (after surgery or resuscitation), genetic colitis (including some related to IL-10 genetic mutations causing infant onset Crohn's disease or TLR4 mutations known to be associated with NEC).

Diamine Oxidase (DAO) Formulations

In embodiments of the present invention, DAO from any source can be used. The DAO can be naturally occurring or synthetic. DAO can be obtained from many sources, including both human and non-human sources. DAO can be isolated from non-human sources such as plants, microorganisms and mammals. DAO can also be expressed in recombinant systems. Recombinant DAO can, by way of example but not limitation, include human DAO expressed in a protein expression system.

Methods for isolating and formulating DAO for administration are well known in the art. The following references describe isolation and preparation of certain DAO formulations.

US 2004/0115189 A1 describes methods for isolating DAO from vegetal sources and is incorporated herein by reference in its entirety.

US 2013/0195830 A1 describes methods for isolating DAO from vegetal sources and perorally applicable formulations and is incorporated herein by reference in its entirety.

US 2013/0344137 A1 describes formulations of DAO for administration and is incorporated herein by reference in its entirety.

US 2014/0212492 A1 also describes formulations of DAO for intestinal delivery and is incorporated herein by reference in its entirety.

U.S. Pat. No. 8,716,244 also describes formulations and delivery methods for DAO and is incorporated herein by reference in its entirety.

U.S. Pat. No. 4,725,540 also describes isolation and formulation of DAO from Candida albicans and is incorporated herein by reference in its entirety.

U.S. Pat. No. 8,722,038 describes isolation of DAO from vegetal sources and is incorporated herein by reference in its entirety.

DAO can be formulated in any form that is acceptable for administration to a neonatal subject. Such formulations can include, by way of example but not limitation, the free form, powder form, lyophilized powder form, a hydrogel, microgranules, microcapsules, nanocapsules or liposomes. DAO can also be obtained from any source that is considered safe for administration, including from plants, microorganisms, animals and the like. For example, but not to otherwise limit this disclosure, DAO can be isolated from pea seedlings, human placenta, Candida crusei and recombinant sources.

The DAO may be in a form naturally-occurring in the organism from which it is obtained, or a recombinant form if expressed in a different organism, or a mutant that retains the activity of DAO. Certain mutations and variants may modulate the safety and/or efficacy profile of DAO and are within the contemplation of the present disclosure. In some embodiments, the DAO is of plant origin. In certain embodiments, the DAO is of non-plant origin. In some embodiments, the DAO is of animal origin. In certain embodiments, the DAO is obtained from a microorganism. In some embodiments, the DAO is obtained from a non-animal, non-plant organism.

In some embodiments, a composition comprising DAO can further comprise a pharmaceutically acceptable diluent. In some embodiments, a composition comprising DAO can further comprise a pharmaceutically acceptable excipient. In some embodiments, a composition comprising DAO but not including human expressed breast milk can further comprise infant formula.

In embodiments where DAO is combined with human expressed breast milk, the DAO can be present at a non-physiological concentration. In certain embodiments, the non-physiological concentration is greater than the physiological concentration of DAO in the human expressed breast milk. In some embodiments, the DAO is added to the human expressed breast milk. In certain embodiments, the amount of DAO present in the compositions or methods described herein is from about 2× to about 500× the physiological concentration of DAO. In other embodiments, the amount of DAO present in the compositions or methods described herein is from about 5× to about 250× the physiological concentration of DAO. In yet another embodiment, the amount of DAO present in the compositions or methods described herein is from about 100× to about 100× the physiological concentration of DAO. In yet another embodiment, the amount of DAO present in the compositions or methods described herein is from about 50× to about 100× the physiological concentration of DAO. In yet another embodiment, the amount of DAO present in the compositions or methods described herein is from about 5× to about 50× the physiological concentration of DAO.

In some embodiments, a method comprises manufacturing infant formula or human milk fortifier, wherein the infant formula or human milk fortifier comprises DAO. In certain embodiments, the manufacturing process comprises a step of adding the DAO to the infant formula or human milk fortifier.

The infant formula can be any infant formula that is administered to infants. By way of example but not limitation, the infant formula can be an infant formula that is exempt or non-exempt by the FDA. Non-limiting examples of such formulas include infant formulas under the names ENFAMIL, SIMILAC, PROLACTA, GERBER, NESTLE and PBM NUTRITIONALS.

In some embodiments, the DAO can be present in a composition or administered at a concentration sufficient to expose the infant to the fetal physiological concentration of DAO. In some embodiments, the DAO can be present in a composition or administered at a concentration sufficient to maintain the circulating DAO level in the infant that was present at birth. In some embodiments, the DAO can be present in a composition or administered at a concentration sufficient for the circulating DAO level in the infant to remain above baseline.

Methods and Timing of Administration

DAO of the present disclosure can be administered to an infant via well-known methods to those of ordinary skill in the art. In some embodiments, the DAO is administered systemically, where systemically is understood to include parenteral and enteral routes of administration. These routes include, by way of example but not limitation, intravenous injection, intravenous infusion, intramuscular injection, subcutaneous injection, mucosal delivery, intranasal administration, transmucosal delivery, pulmonary delivery, oral administration, enteral administration, or rectal administration of an appropriately formulated DAO.

In some embodiments, a DAO-supplemented breast milk is administered enterally. In some embodiments, a DAO-supplemented breast milk is administered by gavage.

In some embodiments, a DAO-supplemented infant formula is administered enterally. In some embodiments, a DAO-supplemented infant formula is administered by gavage.

In certain embodiments, a composition comprising DAO but not including human expressed breast milk is administered systemically. In certain embodiments, the composition is administered enterally. In some embodiments, the composition is administered intravenously. In some embodiments, the composition is administered by gavage.

In some embodiments, the compositions of the present invention can be administered to an infant starting within the first two weeks of life. Administration of the compositions including DAO can continue until it is determined that the risk of colitis is sufficiently reduced or the symptoms, severity or pathology of the condition are reduced.

### EXAMPLES

Unless otherwise noted, the following Materials and Methods were used in Examples 1-5.

### Materials and Methods

Bacterial Preparation.

Klebsiella pneumoniae (ATCC#10031, Manassas, Va.) was incubated in a nutrient broth for 20 hours at 37° C. with agitation at 200 RPM. The bacterial preparation was then centrifuged at 2000×G for 15 minutes. Pellets were resuspended in 5 mL of phosphate buffered saline (PBS) 1×. Bacterial solution was diluted in PBS 1× to a final OD 2.5 at 600 nm, then concentrated 10:1 in PBS 1×, to yield a final bacterial concentration of 10¹⁰ CFU/mL. This concentration was confirmed by serial dilution plate counting.

Dithizone Preparation.

Dithizone solution was prepared the day before each experimental run and kept at −20° C. overnight, and then thawed at room temperature prior to use. 500 μl of 100% ethanol was mixed with 100 μl of ammonium hydroxide and then 60 mg of dithizone powder (43820, Sigma-Aldrich, St. Louis, Mo.) was added to the solution and mixed thoroughly. Another 600 μl of 100% ethanol was added to complete the stock solution of 100 mg/ml. Prior to use this solution was diluted to 1.5 mg/ml. The solution was used for intraperitoneal injection of neonatal mice at a final dose of 33 mg/kgbw.

Diamine Oxidase Preparation and DAO Safety.

Diamine oxidase was obtained from Bio-Research products, EC 1.4.3.6 CAS 9001-53-0 (http://www.bio-researchprod.com/products/dao). A dose of 500 U/kgbw was used for all experiments. Dilutions were with PBS. DAO was administrated systemically to sham operated mice to test safety.

Modified Dithizone/Klebsiella Model.

All animal procedures were conducted in compliance with the protocols approved and authorized by the Institutional Animal Care and Use Committee at the University of Oklahoma Health Sciences Center (protocol number 14-134-1), and were performed in strict accordance with the recommendations in the Guide for the Care and Use of Laboratory Animals. See Care IoLARCo, Animals UoL, Resources NIoHDoR: Guide for the care and use of laboratory animals: National Academies; 1985.

NEC was induced in CD-1 mouse pups (Charles River Laboratory) using the Paneth-cell ablation and Klebsiella infection model, also known as the dithizone/Klebsiella model (DK model), with recommended modifications from the original authors. See Zhang C, Sherman M P, Prince L S, Bader D, Weitkamp J H, Slaughter J C, McElroy S J: Paneth cell ablation in the presence of Klebsiella pneumoniae induces necrotizing enterocolitis (NEC)-like injury in the small intestine of immature mice, Disease models & mechanisms 2012, 5(4):522-532.

Briefly, CD-1 pups were separated from their mothers at 14-16 days old and received an i.p. injection of 33 mg/kg of dithizone (Sigma-Aldrich, St. Louis, Mo.) or an equivalent volume of vehicle alone as control. Six hours after the injection, pups received an enteral gavage of 10⁸ CFU/gram body weight Klebsiella pneumoniae (ATCC#10031, Manassas, Va.) using blunted oral gavage needle (24 gauge×25 mm). Mice were continuously monitored for 10 hours after bacterial gavage and then euthanized for analysis.

Pups were randomly divided into four groups: 1) Sham group, 2) NEC group, 3) NEC with DAO, and 4) DAO alone. DAO was given by i.p. injection of DAO (500 U/kg) at 48 hours, 24 hours, and 1 hour prior to Klebsiella administration.

Histological Evaluation of NEC.

Following incision of the abdomen, the small intestine was visually evaluated for signs of NEC such as intestinal discoloration, intestinal hemorrhage, and intestinal distention. Approximately 1 cm segments were excised and fixed in 10% formalin buffer and stained with hematoxylin and eosin for microscopic examination.

Mucosal injury was evaluated by two blinded pathologists, and graded on a 5-point scale: grade 0, no injury (normal); grade 1, mild separation of lamina propria; grade 2, moderate separation of sub-mucosa; grade 3, severe separation and/or edema in submucosa; grade 4, transmural injury (severe). The analysis was performed on 4-6 μm sections. The final score was based on the area of the most severe injury. NEC is defined as a histological change of grade 2 or more.

In Vivo Intestinal Barrier Function Assay.

To investigate the intestinal lumen to blood permeability barrier function in the NEC model, an in vivo permeability assay was performed using FITC-dextran as previously described (PMID: 24684847). Briefly, all surviving mice pups were gavaged 100 mg/ml 4-kDa FITC-dextran (44 mg/100 g body weight). See Gupta J, Nebreda A R: Analysis of Intestinal Permeability in Mice. Bio-protocol 2014, 4(22):e1289. After four hours, whole blood was collected and centrifuged at 3,000 rpm for 10 minutes. Serum was then diluted with PBS and added in a 96-well black wall microplate in duplicates for measurement of fluorescence intensity using a fluorometer. Standards were included to determine the concentrations of FITC-dextran in serum. High concentrations indicate greater transmucosal transport of FITC-dextran across the intestinal barrier to blood.

Mouse Inflammatory Cytokine Quantification.

Small intestinal samples were homogenized in lysis buffer. Intestinal cytokines were analyzed using Procarta Plex Mouse Cytokine & Chemokine Panel 1A (EPX260-26088-901, eBioscience, San Diego, Calif.) as per manufacturer instructions. Final cytokine levels were normalized to total protein concentration (mg/ml) and reported as pictogram/mg protein).

Statistical Analysis.

Data are presented as mean±SEM and analyzed using one-way ANOVA followed by Dunnett's multiple comparisons test. Survival data was analyzed using Log-rank tests (Mantel-Cox) of Kaplan-Meier Curves. Data was considered significant when p<0.05. All the analysis was performed using GraphPad Prism version 6.00 for Windows, GraphPad Software, La Jolla Calif. USA, www.graphpad.com.

### Example 1

Systemic Administration of DAO Reduced Mortality in DK NEC Model.

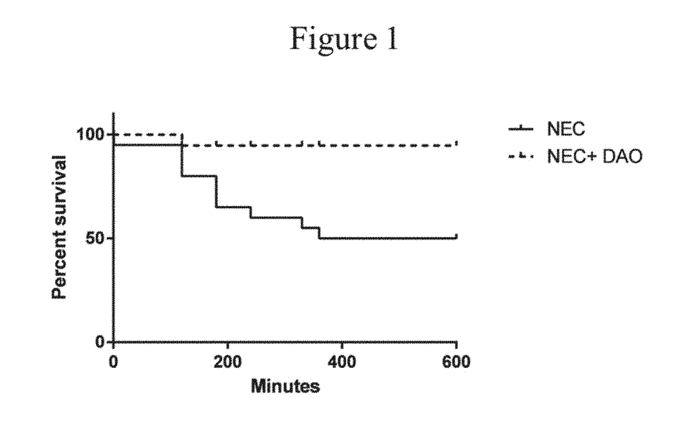

As shown in FIG. 1, we observed a 50% mortality in the NEC group and by pretreating pups with DAO there was a significant reduction in mortality, 5.27% (P=0.0054). In FIG. 1, the survival curve determined with the Kaplan-Meier method was used to evaluate the difference in survival between pups in the NEC group (n=19) and NEC+DAO (n=19). Pretreating pups with DAO significantly improved survival to 94.7%, compared to a mortality of 50% in the NEC group (P=0.0054).

### Example 2

Systemic Administration of DAO Reduced Incidence and Severity of Intestinal Injury in DK NEC Mode.

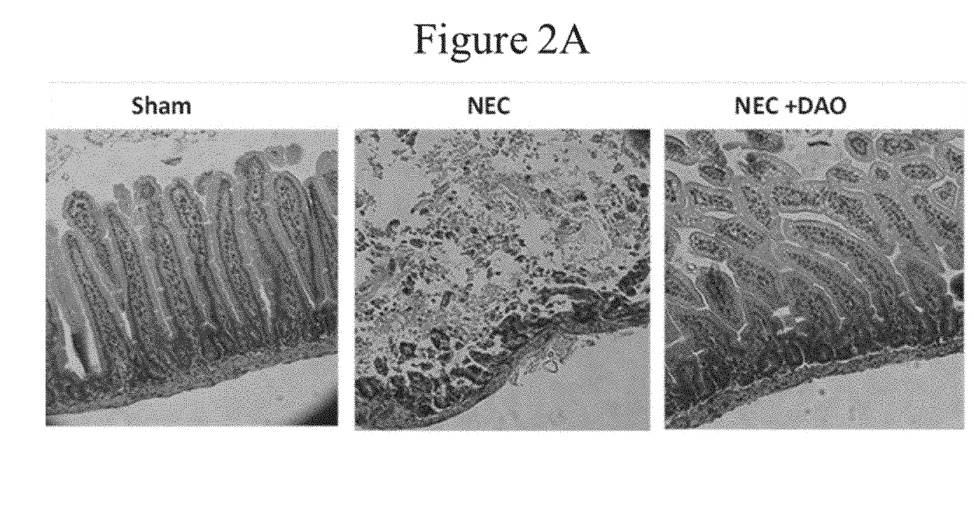

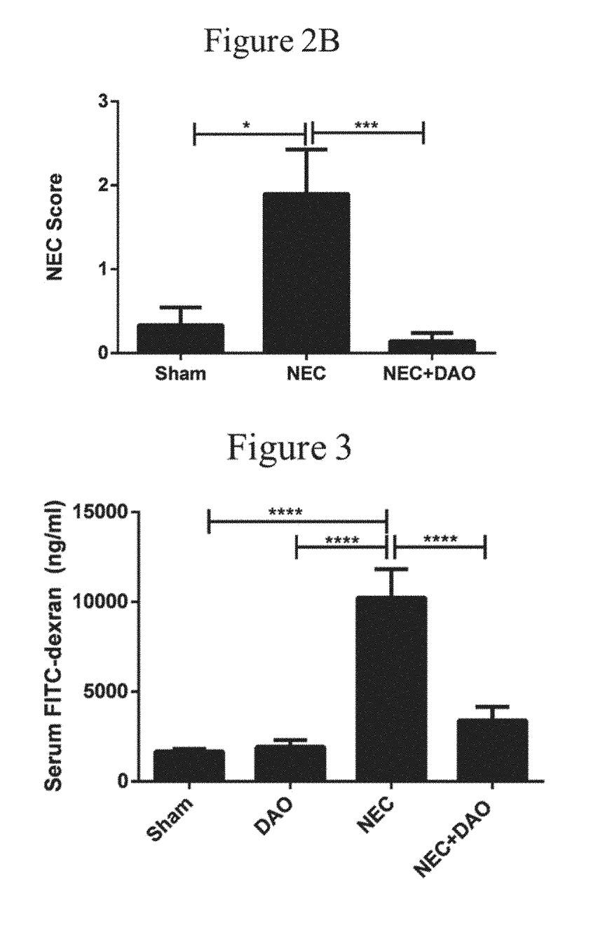

To determine the effect of DAO on the incidence and severity of NEC, sections of the ileum were harvested from the surviving pups and processed for histological examination. Pups in the sham group had healthy villi with normal epithelium and submucosal layers, as shown in FIG. 2A. 50% of pups in the NEC group developed intestinal injury severity scores above 2. A score of ≥2 was defined as histologically-significant NEC. The graph depicted in FIG. 2B represents animals from 2 independent experiments, error bars represent SEM. For FIG. 2B, *P<0.01, ***p<0.001 by one-way ANOVA with Dunnett's multiple comparison test. None of the pups pretreated with DAO had intestinal injury scores above 2, as shown in FIG. 2B, and had scores similar to the shame treated group. This demonstrates that systemic administration of DAO lowers the severity of injury by maintenance of the intestinal architecture.

### Example 3

DAO Administration Attenuated the Increase in Intestinal Permeability in DK NEC Model.

Intestinal permeability was determined through the oral administration of the fluorescent tracer, FITC-dextran (4 kDa), to each group. The concentration of fluorescent FITC-dextran in the blood was then measured. Increased levels correlate directly with the degree of intestinal permeability. As shown in FIG. 3, pups in the NEC group had significantly increased intestinal permeability compared to the sham group. DAO supplementation in NEC pups significantly decreased FITC-dextran flux to blood to near sham levels, indicating that systemic administration of DAO protected intestinal barrier function. Pups in the NEC+DAO group had a twofold decrease in serum FITC-dextran, when compared to the NEC group. Results depicted in FIG. 3 represent mean±SEM from two separate experiments performed in duplicates. ****P<0.0001 by one way ANOVA with Dunnett's multiple comparison test.

### Example 4

Administration of DAO Attenuated Intestinal Cytokine Levels in a DK NEC Model.

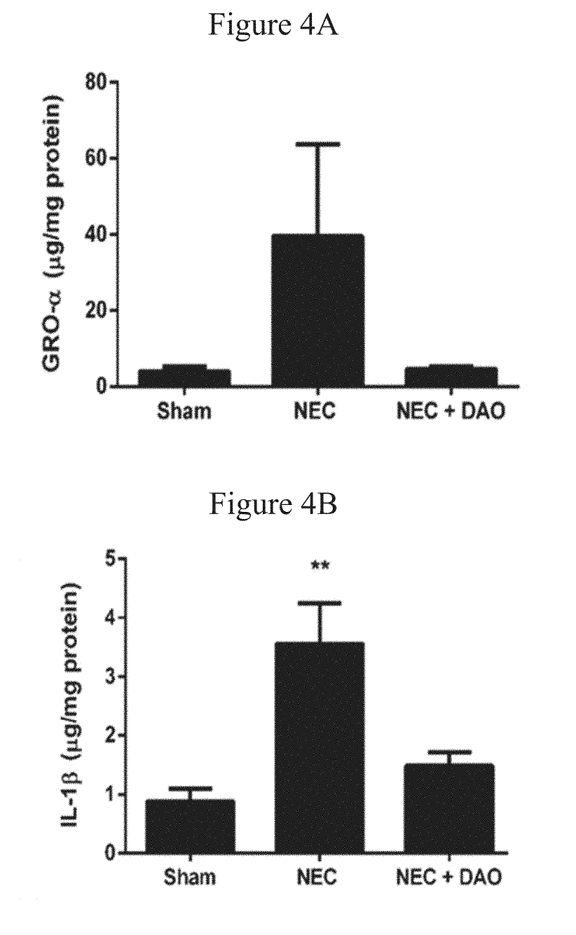

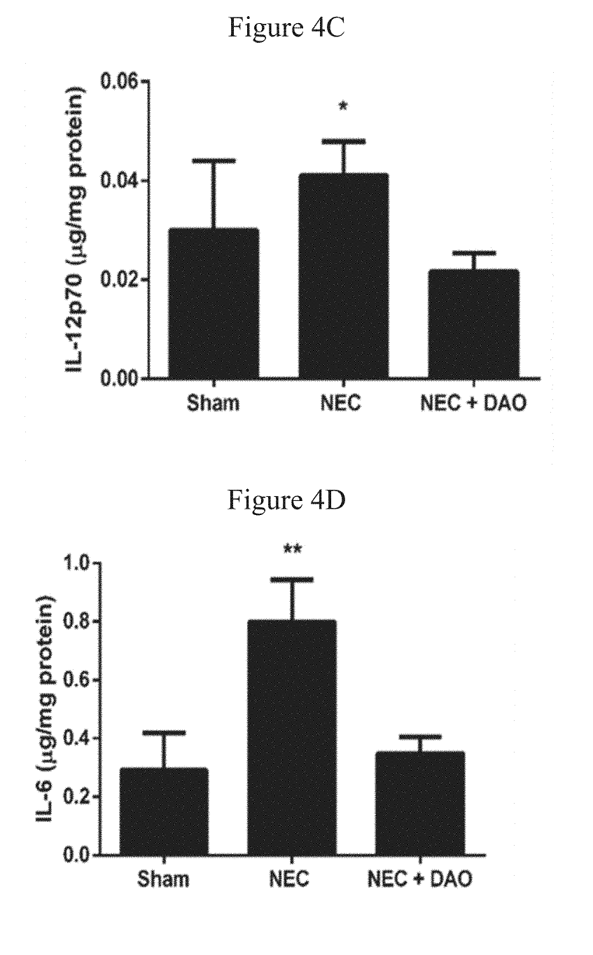

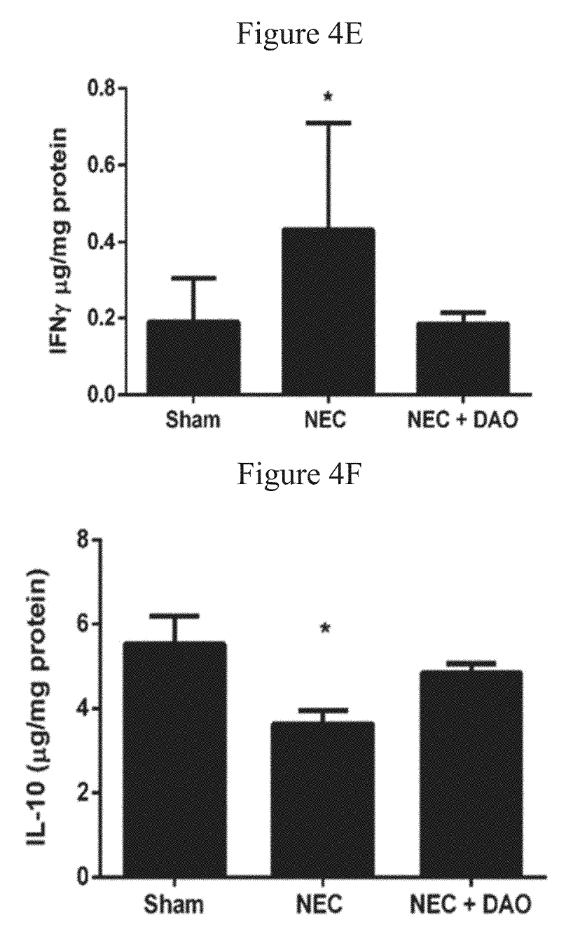

To measure the effect of DAO administration on intestinal inflammation, we measured the protein concentrations of pro-inflammatory cytokines IL-1B, IL-6, IL-12, and IFN-γ from intestinal homogenates of each group. Protein expression of the selected cytokines was determined in the intestinal samples using ProcartaPlex immunoassay. As seen in FIGS. 4A-4F, pups in the NEC+DAO group had a significant 2-4 fold reduction in the levels of IL-6, IL1β, IL-12p70, and INF-γ as compared to the NEC group. Levels of GRO-α were lower in the NEC+DAO group, but did not achieve statistical significance between the groups (P=0.063). The anti-inflammatory cytokine IL-10 was significantly higher in NEC+DAO group as compared the NEC group. For the data described here and in FIGS. 4A-4F, data is represented from two independent experiments, error bars represent SEM, *P<0.01, **P<0.001 by one-way ANOVA with Dunnett's multiple comparison test.

### Example 5

Therapeutic replacement of placental enzyme DAO increases survival, decreases severity of necrotizing enterocolitis in murine model.

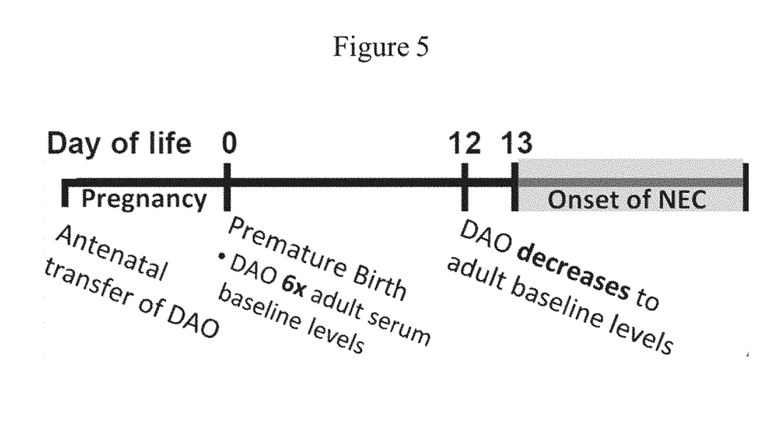

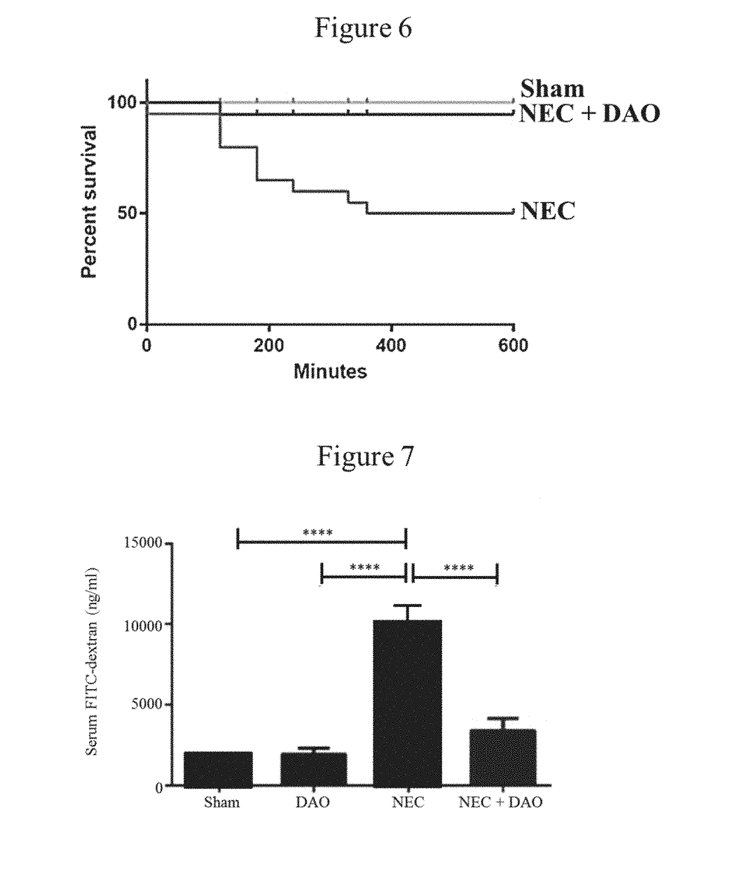

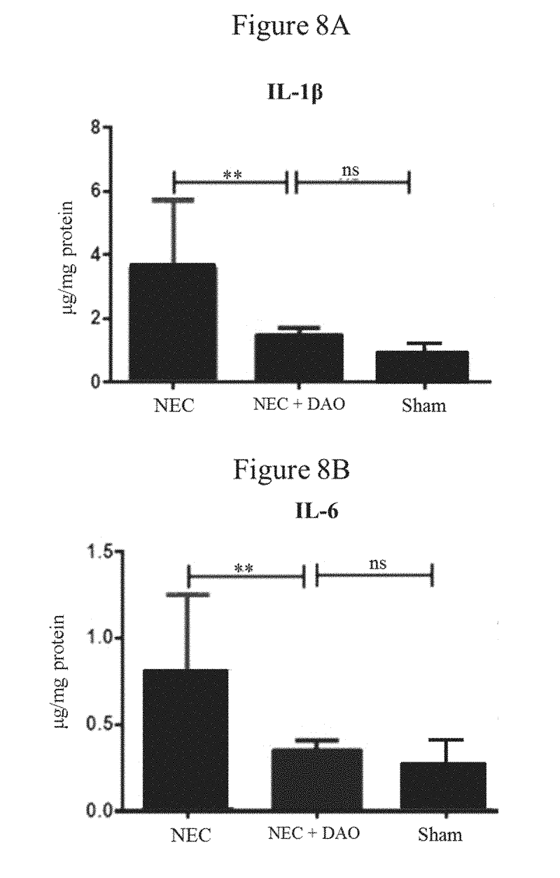

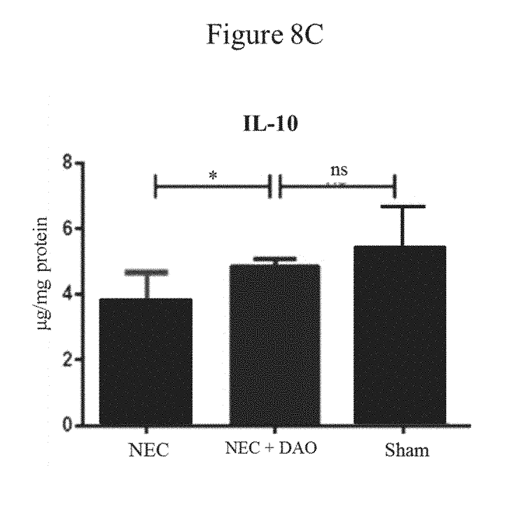

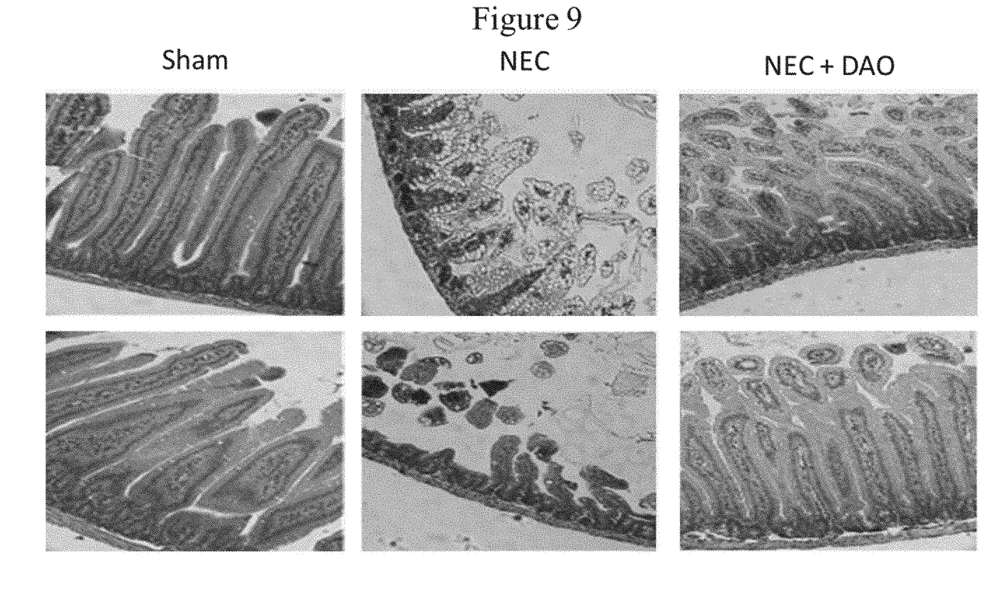

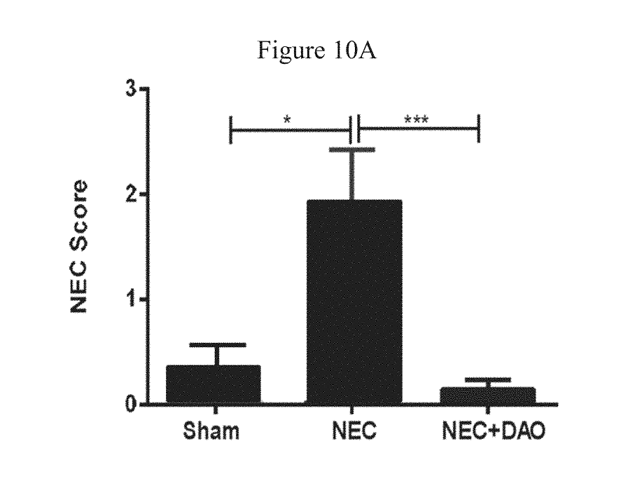

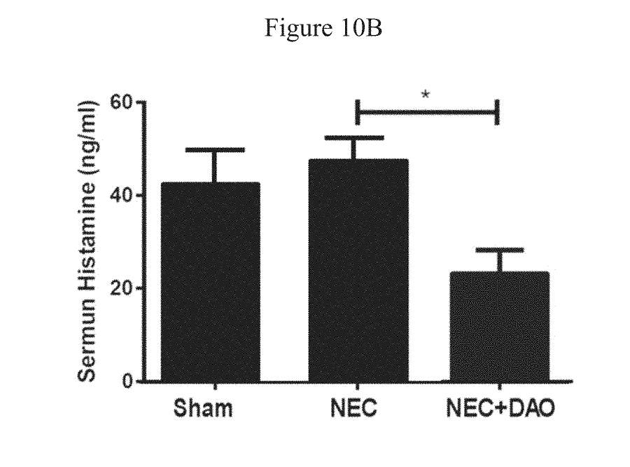

FIGS. 5-10B show the experimental design and results of administering DAO in the DK NEC murine model. DAO was administered IP 48 hrs, 24 hrs, and 1 hr prior to bacterial gavage. In vivo intestinal permeability was determined by measuring serum FITC-dextran. Histological grading was by H&E stain. The serum and tissue cytokine expression was measured by a ProcartaPlex immunoassay. Histamine was measured by ELISA. The results of these experiments show that DAO improves survival, prevents intestinal injury, and maintains integrity in a murine NEC model.

### Example 6

Safety Assessment.

This is a prophetic example. The neonatal necrotizing enterocolitis model in rats is described by Barlow et al. and later modified. See Barlow B, Santulli T V: Importance of multiple episodes of hypoxia or cold stress on the development of enterocolitis in an animal model. Surgery 1975, 77(5):687-690. Jantscher-Krenn E, Zherebtsov M, Nissan C, Goth K, Guner Y S, Naidu N, Choudhury B, Grishin A V, Ford H R, Bode L: The human milk oligosaccharide disialyllacto-N-tetraose prevents necrotising enterocolitis in neonatal rats. Gut 2012, 61(10): 1417-1425. Pregnant time-dated Sprague-Dawley rats are induced at term using Pitocin at a concentration of 1-2 U per animal. Then immediately after birth, pups are randomized into each study group as defined. Animals in the dam-fed (DF) group remain with the dam. All other animals are separated from the dam, housed in a temperature- and humidity controlled incubator and orally gavaged with a special rodent formula (0.2 ml) twice daily.

The formula approximates the protein and caloric content of rat breast milk and consists of 15 g Similac 60/40 (Ross Pediatrics, Columbus, Ohio, USA) in 75 ml of Esbilac canine milk replacer (Pet-Ag, Hampshire, Ill., USA). All animals, dam-fed and gavaged, are exposed to 10 min of hypoxia (5% O2, 95% N2) three times daily in a modular chamber. All animals are sacrificed at 96 hour post-partum; their intestines are collected and inspected with histopathology for the presence of gross necrotic changes or Pneumatosis intestinalis. A 0.5 cm section of the terminal ileum is prepared for H&E staining per standard protocols and scored blindly based on morphological changes that included epithelial sloughing, villus edema, infiltration of neutrophils, apoptosis of villus enterocytes, crypt hyperplasia and misaligned nuclei in the epithelium. If at least one pathology sign is observed, a score of 0.5-1.5 is assigned depending on severity. Two or three signs together result in a score of 2-3. The maximum score of 4 is given in case of complete obliteration of the epithelium with or without intestinal perforation.

Pathology scores are plotted for each animal and the mean calculated per group. A 0.5 cm section of the terminal ileum will be prepared for H&E staining per standard protocols and scored blindly based on morphological changes that included epithelial sloughing, villus edema, infiltration of neutrophils, apoptosis of villus enterocytes, crypt hyperplasia and misaligned nuclei in the epithelium. If at least one pathology sign is observed, a score of 0.5-1.5 will be assigned depending on severity. Two or three signs together result in a score of 2-3. The maximum score of 4 is given in case of complete obliteration of the epithelium with or without intestinal perforation. Each intervention is tested in at least two independent sets of experiments with a total of 8-26 animals per intervention group. Differences between the groups are calculated by one-way ANOVA with the Kruskal-Wallis test and Dunn's multiple comparison tests. Significance is defined as p<0.05.

### Example 7

Evaluation of Oral Administration of DAO in a Rodent Model of Necrotizing Enterocolitis (NEC).

This is a prophetic example. DAO is sourced from Bio-Research Products, Inc. which is a subsidiary of IBEX Technologies, Inc. The laboratory is GMP and ISO 9001:2008 certified. Pregnant time-dated Sprague-Dawley rats (Charles River Labs, Pontage, Mich.) are induced at term using Pitocin at a concentration of 1-2 U per animal. Animals are hand fed for 96 hours with rat milk substitute formula based on cow's milk and free of growth factor. Experimental NEC is induced by asphyxia (breathing 100% nitrogen gas for 60 seconds) and cold stress (4° C. for 10 minutes) twice daily. After 96 hours, all of the surviving animals are killed by euthanasia. Twenty pups per group are needed to achieve 80% power to detect a difference among the treatment groups in survival at a significance level of 0.05 with 25% standard deviation (StatMate software). To determine the most efficient dose, a dose mimicking the human milk physiologic concentration is administered at a concentration of 0.18 u of DAO per mL of formula feed (0.18 u/mL). We are to also administer a high dose, as we did in our prior experiment so we also administer a dose of 18 u of DAO per mL of formula feed (18 u/mL). The administration at the lower dose of DAO mimics the amount of DAO in human breast milk whereas the high dose is a supratherapeutic dose to demonstrate to replicate the high dose administered in the prior experiment. DAO or control (normal saline) is given DAO via gavage starting at birth and then in 8-12-hour intervals until the 8-12 hours prior to euthanasia at 96 hours

Pups are observed and noted for signs of NEC; severe abdominal distension, apnea, cyanosis, diarrhea, and lethargy. Survival rates and timing are recorded for analysis. Pups are euthanized early if under severe distress to reduce suffering. Blood and tissue samples are be collected at spontaneous death or at the termination of the experiment. At the point of terminal collection, the animals are humanely euthanized as in compliance with IACUC standards.

Pups are also evaluated by quantification of plasma and tissue cytokines and oxidative damage. Quantification of serum and tissue levels of cytokines is performed with Luminex 20-plex mouse cytokine kit. Quantification of serum and plasma oxidative damage (total reactive oxygen species and reactive nitrogen species) is performed by OxiSelect™ in-vitro ROS/RNS Assay Kit. Quantification of plasma histamine levels. Quantification of plasma levels is performed with the Oxford Biomedical Research ELISA.

The present disclosure provides compositions and methods well adapted to attain the ends and advantages mentioned as well as those that are inherent therein. While numerous changes can be made by those skilled in the art, such changes are encompassed within the spirit of this invention as illustrated, in part, by the appended claims.

What is claimed is:

1. A method for preventing or treating colitis in an infant in need thereof, comprising:
administering a diamine oxidase (DAO)-supplemented formula to the infant, wherein the DAO-supplemented formula comprises DAO in an amount in excess of any amount of DAO naturally present in the formula.

2. The method of claim 1, wherein the DAO-supplemented formula comprises human expressed breast milk.

3. The method of claim 2, wherein the DAO is present at a concentration greater than the physiological concentration of DAO in the human expressed breast milk.

4. The method of claim 1, wherein the DAO-supplemented formula does not comprise human breast milk.

5. The method of claim 1, wherein the DAO is of non-human origin.

6. The method of claim 1, wherein the infant is a premature infant.

7. The method of claim 1, wherein the colitis is necrotizing enterocolitis (NEC).

8. The method of claim 1, wherein the DAO-supplemented formula further comprises a human milk fortifier.

9. A method for preventing loss of intestinal integrity in an infant in need thereof, comprising:
administering a diamine oxidase (DAO)-supplemented formula to the infant, wherein the DAO-supplemented formula comprises DAO in an amount in excess of any amount of DAO naturally present in the formula.

10. The method of claim 9, wherein the DAO-supplemented formula comprises human expressed breast milk.

11. The method of claim 10, wherein the DAO is present at a concentration greater than the physiological concentration of DAO in the human expressed breast milk.

12. The method of claim 9, wherein the DAO-supplemented formula does not comprise human breast milk.

13. The method of claim 9, wherein at least a portion of the DAO is of non-human origin.

14. The method of claim 9, wherein the infant is a premature infant.

15. The method of claim 9, wherein the DAO-supplemented formula further comprises a human milk fortifier.

16. A composition comprising human expressed breast milk and diamine oxidase (DAO), wherein said DAO is present at a non-physiological concentration.

17. The composition of claim 16, wherein the DAO is present at a concentration greater than the physiological concentration of DAO in the human expressed breast milk.

18. The composition of claim 16, wherein the concentration of DAO in the DAO-supplemented breast milk is greater than a maximum physiological concentration of DAO in the human expressed breast milk.

19. The composition of claim 16, wherein at least a portion of the DAO is of non-human origin.

20. The composition of claim 16, further comprising a human milk fortifier.
